(19) United States
(12) Patent Application Publication
(10) Pub. No.: US 2024/0425863 A1
(43) Pub. Date: Dec. 26, 2024

(54) USE OF PI3KC2B INHIBITORS FOR THE PRESERVATION OF VASCULAR ENDOTHELIAL CELL BARRIER INTEGRITY

(71) Applicants: INSERM (INSTITUT NATIONAL DE LA SANTÉ ET DE LA RECHERCHE MÉDICALE), Paris (FR); UNIVERSITÉ PAUL SABATIER TOULOUSE III, Toulouse (FR); UNIVERSITY COLLEGE LONDON, London (GB); CENTRE HOSPITALIER UNIVERSITAIRE DE TOULOUSE, Toulouse (FR); UNIVERSITÉ DE CAEN NORMANDIE, Caen (FR); CENTRE HOSPITALIER RÉGIONAL UNIVERSITAIRE DE CAEN, Caen (FR)

(72) Inventors: Marie-Pierre GRATACAP, Toulouse Cedex 4 (FR); Jean DARCOURT, Toulouse Cedex 4 (FR); Bart VANHAESEBROECK, London (GB); Gaëtan CHICANNE, Toulouse Cedex 4 (FR); Bernard PAYRASTRE, Toulouse Cedex 4 (FR); Vincent LARRUE, Toulouse Cedex 4 (FR); Romain SOLINHAC, Toulouse Cedex 4 (FR); Aude JAFFRE, Toulouse Cedex 4 (FR); Denis VIVIEN, Caen (FR); Typhaine ANQUETIL, Toulouse Cedex 4 (FR)

(21) Appl. No.: 18/634,158

(22) Filed: Apr. 12, 2024

Related U.S. Application Data
(63) Continuation of application No. 18/055,597, filed on Nov. 15, 2022, now abandoned.
Application No. 18/055,597 is a division of application No. 16/972,152, filed on Dec. 4, 2020, now Pat. No. 12,123,000.

(30) Foreign Application Priority Data
Jun. 15, 2018 (EP) 18305735.5

(51) Int. Cl. C12N 15/113; A61P 9/10; G01N 33/50

(52) U.S. Cl. CPC C12N 15/1137; A61P 9/10; G01N 33/5064; C12N 2310/14; C12N 2310/531

(57) ABSTRACT

Ischemic conditions are a leading cause of death for both men and women, Ischemia, a condition characterized by reduced blood flow and oxygen to an organ. Re-establishment of blood flow, or reperfusion, and re-oxygenation of the affected area following an ischemic episode is critical to limit irreversible damage. However, reperfusion also associates potentially damaging consequences. For instance, increased vascular permeability is an important contributor to edema and tissue damage following ischemic events. Here the inventors shows that genetic inhibition of PI3K-C2β reduces cerebral infarction in two ischemic/reperfusion (I/R) models and improves neurological outcome. The genetic inhibition stabilizes the blood-brain barrier (BBB) after ischemic stroke and reduces inflammation. Accordingly, the present invention relates to a method for the preservation of vascular endothelial cell barrier integrity in a patient in need thereof comprising administering to the subject a therapeutically effective amount of a PI3KC2β inhibitor.

### CROSS-REFERENCE TO RELATED APPLICATIONS

This application is a Continuation application of U.S. Ser. No. 18/055,597 filed on Nov. 15, 2022, which is a Divisional application of U.S. Ser. No. 16/972,152 filed on Dec. 4, 2020, which was a national stage application under Rule 371 of PCT/EP2019/065694 filed Jun. 14, 2019, which claimed priority to European Application 18305735.5 filed Jun. 15, 2018.

### SEQUENCE LISTING

This application includes as the Sequence Listing the complete contents of the XML file “2022-11-15_11450654US2_seqlisting” created Nov. 15, 2022 containing 3,576 bytes, hereby incorporated by reference.

### FIELD OF THE INVENTION:

The present invention relates to the use of PI3KC2β inhibitors for the preservation of vascular endothelial cell barrier integrity.

### BACKGROUND OF THE INVENTION:

Ischemic conditions are a leading cause of death for both men and women. Ischemia, a condition characterized by reduced blood flow and oxygen to an organ. For instance ischemic injuries may occur in various organs and tissues, including the heart, which can lead to myocardial infarction and the brain, which can lead to stroke ischemia. Re-establishment of blood flow, or reperfusion, and re-oxygenation of the affected area following an ischemic episode is critical to limit irreversible damage. However, reperfusion also associates potentially damaging consequences. For instance, increased vascular permeability is an important contributor to edema and tissue damage following ischemic events. Development of edema determines disruption of integrity which is detrimental to recovery and also permits extravasation of fibronectin and fibrinogen that form the provisional matrix network used by leukocytes for infiltrating. Vascular damage also contributes to the no-reflow phenomenom which is observed in 30% of patients with a reperfused anterior wall myocardial ischemia and is associated with a higher incidence of death. Leakiness of blood vessels in the tissues therefore contributes to disease progression. The prevalence of ischemic conditions necessitates the development of therapies and therapeutic agents that can effectively prevent, reduce, or counteract ischemia and ischemia-reperfusion injury. Thus, there is a significant need for new and more effective therapies and therapeutic agents for the treatment of ischemia and ischemia-reperfusion injuries.

### SUMMARY OF THE INVENTION

The present invention relates to the use of PI3KC2β inhibitors for the preservation of vascular endothelial cell barrier integrity. In particular, the present invention is defined by the claims.

### DETAILED DESCRIPTION OF THE INVENTION

The first object of the present invention relates to a method for the preservation of vascular endothelial cell barrier integrity in a patient in need thereof comprising administering to the subject a therapeutically effective amount of a PI3KC2β inhibitor.

As used herein, the term “vascular endothelial cell barrier” refers to the layer of cells that line the interior surface of blood vessels and act as a selective barrier between the vessel lumen and surrounding tissue, by controlling the transit of fluids, materials and cells such as myeloid cells and white blood cells into and out of the bloodstream. Excessive or prolonged increases in permeability of vascular endothelial cell barrier leads to tissue oedema/swelling. Accordingly the term “preservation of vascular endothelial cell barrier integrity” means the maintenance of the vascular endothelial cell barrier by avoiding or limiting permeability of said barrier.

In some embodiments, the PI3KC2β inhibitor of the present invention is particularly suitable for the preservation of vascular endothelial cell barrier integrity during sepsis. As used herein, the term “sepsis” has its general meaning in the art and represents a serious medical condition that is characterized by a whole-body inflammatory state. In addition to symptoms related to the provoking infection, sepsis is characterized by presence of acute inflammation present throughout the entire body, and is, therefore, frequently associated with fever and elevated white blood cell count (leukocytosis) or low white blood cell count and lower-than-average temperature, and vomiting. In particular, sepsis is defined as a deregulated immune response to infection, translating into life-threatening organs dysfunction, defined by a Sequential Organ Failure Assessment score of 2 more. Infection can be suspected or proven, or a clinical syndrome pathognomonic for infection. Septic shock is defined by infection and the need for vasopressors to maintain mean blood pressure ≥65 mmHg and arterial lactate levels >2 mmol/l.

In some embodiments, the PI3KC2β inhibitor of the present invention is particularly suitable for the preservation of vascular endothelial cell barrier integrity during the treatment of ischemic conditions.

As used herein, the term “ischemic condition” has its general meaning in the art and refers to any condition that result from ischemia. As used herein, the term “ischemia” as used herein refers to a restriction in blood supply with resultant damage or dysfunction of the organ. Rather than hypoxia (a more general term denoting a shortage of oxygen, usually a result of lack of oxygen in the air being breathed), ischemia is an absolute or relative shortage of the blood supply to an organ, i.e. a shortage of oxygen, glucose and other blood-borne components. For example ischemic conditions include but are not limited to renal ischemia, retinal ischemia, brain ischemia and myocardial ischemia. More particularly, the term includes but it is not limited to coronary artery bypass graft surgery, global cerebral ischemia due to cardiac arrest, focal cerebral infarction, carotid stenosis or occlusion leading to cerebral ischemia, cardiogenic thromboembolism, stroke, spinal stroke and spinal cord injury.

In some embodiments, the method of the present invention is particularly suitable for the treatment of an acute ischemic stroke. As used herein, the term “acute ischemic stroke” or ‘AIS” refers to those patients having or at risk for “definite acute ischemic cerebrovascular syndrome (AICS)” as defined by the diagnostic criteria of Kidwell et al. “Acute Ischemic Cerebrovascular Syndrome: Diagnostic Criteria,” Stroke, 2003, 34, pp. 2995-2998 (incorporated herein by reference). Accordingly, acute ischemic stroke refers to an acute onset of neurologic dysfunction of any severity consistent with focal brain ischemia.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the PI3KC2β inhibitor of the present invention is particularly suitable for reducing infarct size, preventing or reducing edema, preventing hemorrhage and preventing no-reflow. As used herein, the term “no-reflow” has been increasingly used in published medical reports to describe microvascular obstruction and reduced flow after opening an occluded artery. In its broadest meaning, the term “preventing no-reflow” or “prevention of no-reflow” refers to reducing or avoiding the no-reflow.

In some embodiments, the PI3KC2β inhibitor of the present invention is particularly suitable for preventing ischemia-reperfusion injuries. As used herein, the term “reperfusion” has its general meaning in the art and refers to the restoration of blood flow to a tissue following ischemia. Accordingly, the term “ischemia reperfusion” is thus intended to encompass an event wherein an episode of ischemia is followed by an episode of reperfusion and the term “ischemia reperfusion injury” refers to the tissue damage caused by an ischemia reperfusion event.

In some embodiments, the method of the present invention is performed sequentially or concomitantly with a standard method for treating ischemic conditions. Typically, standard methods include reperfusion of the ischemic organ by angioplasty, thrombolysis, or surgical thrombectomy. The term “thrombolysis” means the administration of thrombolytic agents. Typically thrombolysis involves the use of t-PA. As used herein, the term “t-PA” has its general meaning in the art and refers to tissue-type plasminogen activator. The term includes native t-PA and recombinant t-PA, as well as modified forms of t-PA that retain the enzymatic or fibrinolytic activities of native t-PA. The enzymatic activity of t-PA can be measured by assessing the ability of the molecule to convert plasminogen to plasmin. The fibrinolytic activity of t-PA may be determined by any in vitro clot lysis activity known in the art. Recombinant t-PA has been described extensively in the prior art and is known to the person of skill. t-PA is commercially available as alteplase (Activase® or Actilyse®). Modified forms of t-PA (“modified t-PA”) have been characterized and are known to those skilled in the art. Modified t-PAs include, but are not limited to, variants having deleted or substituted amino acids or domains, variants conjugated to or fused with other molecules, and variants having chemical modifications, such as modified glycosylation. Several modified t-PAs have been described in PCT Publication No. WO93/24635; EP 352,119; EP382174. In some embodiments, the modified form of t-PA is Tenecteplase. As used herein, the term “tenecteplase,” also known as TNK-t-PA or TNKASE™ brand of tissue-plasminogen activator variant, refers to a t-PA variant designated T103N, N117Q, K296A, H297A, R298A, R299A t-PA available from Genentech, Inc. (South San Francisco Calif.) wherein Thr103 of wild-type t-PA is changed to Asn (T103N), Asn 117 of wild-type t-PA is changed to Gln (N117Q), and Lys-His-Arg-Arg 296-299 of wild-type t-PA is changed to Ala-Ala-Ala-Ala (KHRR296-299AAAA). Tenecteplase is a genetically engineered variant of human t-PA cloned and expressed in Chinese hamster ovary cells (see Keyt et al., Proc. Natl. Acad. Sci USA, 91:3670-3674 (1994) and Verstraete, Am. J. Med, 109:52-58 (2000) for an overview of third-generation thrombolytic drugs in general). Tenecteplase was engineered to have increased fibrin specificity and an increased half-life compared to alteplase.

In some embodiments, the present invention relates to method of treating an ischemic condition in a patient in need thereof comprising the steps consisting of i) restoring blood supply in the ischemic tissue, and preserving the vascular endothelial cell barrier integrity of said ischemic tissue by administering to said patient a therapeutically effective amount of PI3KC2β inhibitor.

As used herein, the term “PI3KC2β” has its general meaning in the art and refers to the phosphatidylinositol 4-phosphate 3-kinase C2 domain-containing subunit beta, encoded by the PIK3C2B gene (Gene ID: 5287). The protein belongs to the phosphoinositide 3-kinase (PI3K) family and contains a lipid kinase catalytic domain as well as a C-terminal C2 domain, a 25 characteristic of class II PI3-kinases. C2 domains act as calcium-dependent phospholipid binding motifs that mediate translocation of proteins to membranes, and may also mediate protein-protein interactions. The term is also known as C2-PI3K or phosphoinositide 3-kinase-C2-beta. An exemplary human amino acid sequence is represented by SEQ ID NO:1.

>sp|O00750|P3C2B_HUMAN Phosphatidylinositol
4-phosphate 3-kinase C2 domain-containing
subunit beta OS = Homo sapiens OX = 9606
GN = PIK3C2B PE = 1 SV = 2
SEQ ID NO: 1
MSSTQGNGEHWKSLESVGISRKELAMAEALQMEYDALSRLRHDKEENRA
KQNADPSLISWDEPGVDFYSKPAGRRTDLKLLRGLSGSDPTLNYNSLSP
QEGPPNHSTSQGPQPGSDPWPKGSLSGDYLYIFDGSDGGVSSSPGPGDI
EGSCKKLSPPPLPPRASIWDTPPLPPRKGSPSSSKISQPSDINTFSLVE
QLPGKLLEHRILEEEEVLGGGGQGRLLGSVDYDGINDAITRLNLKSTYD
AEMLRDATRGWKEGRGPLDFSKDTSGKPVARSKTMPPQVPPRTYASRYG
NRKNATPGKNRRISAAPVGSRPHTVANGHELFEVSEERDEEVAAFCHML
DILRSGSDIQDYFLTGYVWSAVTPSPEHLGDEVNLKVTVLCDRLQEALT
FTCNCSSTVDLLIYQTLCYTHDDLRNVDVGDFVLKPCGLEEFLQNKHAL
GSHEYIQYCRKFDIDIRLQLMEQKVVRSDLARTVNDDQSPSTLNYLVHL
QERPVKQTISRQALSLLFDTYHNEVDAFLLADGDFPLKADRVVQSVKAI
CNALAAVETPEITSALNQLPPCPSRMQPKIQKDPSVLAVRENREKVVEA
LTAAILDLVELYCNTFNADFQTAVPGSRKHDLVQEACHFARSLAFTVYA
THRIPIIWATSYEDFYLSCSLSHGGKELCSPLQTRRAHFSKYLFHLIVW
DQQICFPVQVNRLPRETLLCATLYALPIPPPGSSSEANKQRRVPEALGW
VTTPLFNFRQVLTCGRKLLGLWPATQENPSARWSAPNFHQPDSVILQID
FPTSAFDIKFTSPPGDKFSPRYEFGSLREEDQRKLKDIMQKESLYWLTD
ADKKRLWEKRYYCHSEVSSLPLVLASAPSWEWACLPDIYVLLKQWTHMN
HQDALGLLHATFPDQEVRRMAVQWIGSLSDAELLDYLPQLVQALKYECY
LDSPLVRFLLKRAVSDLRVTHYFFWLLKDGLKDSQFSIRYQYLLAALLC
CCGKGLREEFNRQCWLVNALAKLAQQVREAAPSARQGILRTGLEEVKQF
FALNGSCRLPLSPSLLVKGIVPRDCSYFNSNAVPLKLSFQNVDPLGENI
RVIFKCGDDLRQDMLTLQMIRIMSKIWVQEGLDMRMVIFRCFSTGRGRG
MVEMIPNAETLRKIQVEHGVTGSFKDRPLADWLQKHNPGEDEYEKAVEN
FIYSCAGCCVATYVLGICDRHNDNIMLKTTGHMFHIDFGRFLGHAQMFG
NIKRDRAPFVFTSDMAYVINGGDKPSSRFHDFVDLCCQAYNLIRKHTHL
FLNLLGLMLSCGIPELSDLEDLKYVYDALRPQDTEANATTYFTRLIESS
LGSVATKLNFFIHNLAQMKFTGSDDRLTLSFASRTHTLKSSGRISDVFL
CRHEKIFHPNKGYIYVVKVMRENTHEATYIQRTFEEFQELHNKLRLLFP
SSHLPSFPSRFVIGRSRGEAVAERRREELNGYIWHLIHAPPEVAECDLV
YTFFHPLPRDEKAMGTSPAPKSSDGTWARPVGKVGGEVKLSISYKNNKL
FIMVMHIRGLQLLQDGNDPDPYVKIYLLPDPQKTTKRKTKVARKTCNPT
YNEMLVYDGIPKGDLQQRELQLSVLSEQGFWENVLLGEVNIRLRELDLA
QEKTGWFALGSRSHGTL

As used herein, a “PI3KC2β inhibitor” refers to any compound natural or not which is capable of inhibiting the activity of PI3KC2β, in particular PI3KC2β kinase activity. The term encompasses any PI3KC2B inhibitor that is currently known in the art or that will be identified in the future, and includes any chemical entity that, upon administration to a patient, results in inhibition or down-regulation of a biological activity associated with activation of the PI3KC2β. The term also encompasses inhibitor of expression. In some embodiments, the PI3KC2β inhibitor is selective over the other kinases. By “selective” it is meant that the inhibition of the selected compound is at least 10-fold, preferably 25-fold, more preferably 100-fold, and still preferably 300-fold higher than the inhibition of the other PI3K kinases. The PI3KC2β inhibition of the compounds may be determined using various methods well known in the art.

In some embodiments, the PI3KC2β inhibitor is a small organic molecule.

In some embodiments, the PI3KC2β inhibitor is an inhibitor of PI3KC2β expression. An “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of PI3KC2β mRNA by binding thereto and thus preventing protein translation or increasing mRNA degradation, thus decreasing the level of PI3KC2β, and thus activity, in a cell. For example, antisense oligonucleotides of at least about 15 bases and complementary to unique regions of the mRNA transcript sequence encoding PI3KC2β can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. PI3KC2β gene expression can be reduced by contacting a subject or cell with a small double stranded RNA (dsRNA), or a vector or construct causing the production of a small double stranded RNA, such that PI3KC2β gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing PI3KC2β. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

According to the invention, the PI3KC2β inhibitor is administered to the patient in a therapeutically effective amount. By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (e.g. PI3KC2β inhibitor) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

A further aspect of the invention relates to a method for screening a plurality of test substances useful for the treatment of an ischemic condition in a patient in need thereof comprising the steps consisting of (a) testing each of the test substances for its ability to inhibit the activity or expression of PI3KC2β and (b) and positively selecting the test substances capable of said inhibition.

In some embodiments, the screening method of the present invention comprises the step of (i) providing a PI3KC2β protein; (ii) contacting the PI3KC2β protein with a test substance wherein the substance is expected to inhibit the kinase activity of the PI3KC2β protein; and (iii) selecting a test substance as a candidate that decreases the kinase activity of PI3KC2β in comparison to a negative control that is not contacted with a test substance.

Typically, PI3KC2β protein come from various sources and sequences in the art may be used for the present disclosure as long as it contains a kinases activity. In one embodiment, a full or partial length of PI3KC2β can be used (e.g. SEQ ID NO:1).

In some embodiments, PI3KC2β protein is provided as a cell that endogenously or exogenously express the protein. For example, mammalian cells are prepared to express the protein of interest such as PI3KC2β through a transient or stable transfection or cells that endogenously express the protein of interest may be used. Cells endogenously expressing PI3KC2β may include but is not limited to endothelial cells. The cells obtained may be cultured in a cell culture dish and treated with a test substance for a certain period time in a suitable medium, from which the whole proteins are extracted and tested/detected for kinase activity of PI3KC2β protein. Alternatively established cell lines may be used, in which case the cells are transfected with a plasmid expressing PI3KC2β. The example of such cells include but is not limited to 293, 293T or 293A (Graham F L, Smiley J, Russell W C, Nairn R (July 1977). “Characteristics of a human cell line transformed by DNA from human adenovirus type 5”. J. Gen. Virol. 36(1):59-74; and Louis N, Evelegh C, Graham F L (July 1997). “Cloning and sequencing of the cellular-viral junctions from the human adenovirus type 5 transformed 293 cell line”. Virology 233 (2): 423-9).

The term “test substance” refers generally to a material that is expected to decrease, reduce, suppress or inhibit the kinase activity of PI3KC2β, which include small molecules, high molecular weight molecules, mixture of compounds such as natural extracts or cell or tissue culture products, biological material such as proteins, antibodies, peptides, DNA, RNA, antisense oligonucleotides, RNAi, aptamer, RNAzymes and DNAzymes, or glucose and lipids, but is not limited thereto. The test substances may be polypeptides having amino acid residues of below 20, particularly 6, 10, 12, 20 aa or above 20 such as 50aa. These materials are obtained from synthetic or natural compound libraries and the methods to obtain or construct libraries are known in the art. For example, synthetic chemical library may be obtained from Maybridge Chemical Co. (UK), Comgenex (USA), Brandon Asociates (USA), Microsource (USA) and Sigma-Aldrich (USA). The chemical library of natural origin may be obtained from Pan Laboratories (USA) and MycoSearch (USA). Further test substances may be obtained by various combinatorial library construction methods known in the art including for example, biological libraries, spatially addressable parallel solid phase or solution phase libraries. Test substance of a library may be composed of peptides, peptoides, circular or liner oligomeric compounds, template based compounds such as benzodiazepine, hydantoin, biaryls, carbocyclic and polycyclic compounds such as naphthalene, phenothiazine, acridine, steroids and the like, carbohydrate and amino acid derivatives, dihydropyridine, benzhydryl and heterocyclic compounds such as triazine, indole, thiazolidine and the like, but does not limited thereto.

The invention will be further illustrated by the following figures and examples. However, these examples and figures should not be interpreted in any way as limiting the scope of the present invention.

### FIGURES

FIGS. 1A-1D Genetic inhibition of PI3K-C2β reduces cerebral infarction in two ischemia/reperfusion (I/R) models and improves neurological outcome. (A) Graph quantification of infarct volume measurements at 24 hours after thromboembolic stroke in wild-type (WT) and C2β^{D1212A/D1212A} mice showing a smaller ischemic lesion in the C2β^{D1212A/D1212A} animals (PI3KC2β KI mice) (n=17-19 mice per group; ***P<0.001, unpaired t-test). (B) Graph quantification of infarct volume measurements in wild-type (WT), heterozygous (C2β^{BWT/D1212A}) and homozygous (C2β^{D1212A/D1212A}) mice subjected to transient middle cerebral artery occlusion (tMCAO) for 1 h followed by 24 h reperfusion (n=14-27 mice per group). **P<0.01; ***P<0.001, unpaired t-test. SHAM: operated WT mice without monofilament insertion. Results show a smaller ischemic lesion according to the level of PI3KC2β inhibition. (C) Mortality evaluation of 10-week-old C2β^{D1212A/D1212A} and WT mice between day 0 and day 1 after tMCAO showing a better survival when PI3KC2β is inactive (n=30-39 per group). (D) Neurological scores evaluated at 24 h by Bederson (left panel) and Grip (right panel) test based on a five point system (n=27 mice per group; *P<0.05, Mann-Whitney test).

FIGS. 2A-2E Genetic inhibition of PI3K-C2β stabilizes the blood-brain barrier (BBB) after ischemic stroke and reduces inflammation. Leakage of Evans blue dye in brain parenchyma (A-B) Graph quantification of Evans blue dye extravasation measurements in wild-type (WT) and PI3KC2β KI (C2β^{D1212A/D12124}) mice subjected to transient middle cerebral artery occlusion (tMCAO) for 1 h followed by 24 h reperfusion (n=10-16 mice per group). **P<0.01; Mann Whitney test. (C) Genetic inhibition of PI3K-C2β reduces inflammation—Relative gene expression of interleukin-1β (IL-1β), interleukin-6 (IL-6) and tumor necrosis factor (TNFα) 24 hours following tMCAO in the cortex and basal ganglia of control (WT) and PI3KC2β KI (C2β^{D1212A/D1212A}) mice. The mRNA levels are given as the fold increase normalized to rps29 relative to the corresponding contralateral hemisphere; TNFα (n=10-14 mice per group); IL-1β, IL-6 (n=8-10 mice per group). Data represent mean±SEM, **P<0.01; ***P=0.001; Unpaired t-test with Welch's correction. (D) Genetic inhibition of PI3K-C2β reduces neutrophils recruitment—Graph quantification for neutrophils infiltration in the ischemic hemisphere of wild-type (WT) and PI3KC2β KI (C2β^{D1212A/D1212A}) mice subjected to tMCAO for 1 h followed by 24 h reperfusion (n=6 mice per group). **P<0.01; Mann Whitney test. (E) Representative T2-weighted MRI of WT and C2β^{D1212A/D1212A} mice taken 24 hours after the onset of in situ clot formation by alpha thrombin, and graph quantification of area stained by P-Selectin using MPIOs in ipsilateral normalize to contralateral cortex in percentage (n=10-11 mice per group; *P<0.05; Unpaired t-test with Welch's correction).

FIGS. 3A-3C Role of endothelial versus hematopoietic PI3K-C2β in its neuroprotective effects. (A) Inhibition of endothelial PI3KC2β reduces cerebral infarction-Graph quantification of infarct volume measurements in bone marrow (BM) chimeric mice one day after tMCAO (n=13-16 mice per group) **P<0.01; ***P<0.001, unpaired t-test. WT>WT: transplantation of WT BM into WT hosts; C2β^{D1212A/D1212A}>WT: transplantation of PI3K-C2β KI BM into WT hosts; C2β^{D1212A/D1212A}>C2β^{D1212A/D1212A}: transplantation of PI3K-C2β KI BM into PI3K-C2β KI hosts; WT>C2β^{D1212A/D1212A}: transplantation of WT BM into PI3K-C2β KI hosts. A stronger protection was observed when WT BM was transplanted into PI3K-C2β KI hosts (WT>KI) suggesting that the inhibition of non hematopoietic PI3K-C2β (likely endothelial) was critical to protect from ischemic stroke lesions. (B) Inhibition of endothelial PI3KC2β reduces edema-Edema volume in chimeric mice 24 hours after tMCAO (n=13 mice per group). **P<0.01 vs WT controls; Unpaired t-test. (C) Inhibition of endothelial PI3KC2β reduces neutrophils infiltration—graph quantification for neutrophils infiltration in the ischemic hemisphere in the indicated groups 24 hours after tMCAO (WT>WT n=6; KI>WT n=4; KI>KI n=7; WT>KI n=5; Mann Whitney test).

FIGS. 4A-4C Impact of PI3K-C2β knock-down on endothelial (hCMEC/D3 cells) monolayer permeability. (A) PI3K-C2β is critical for the regulation of PI3P level in endothelial cells (1) Lysates from human brain capillary endothelial cells (hCMEC/D3) transduced with shRNA control (Sh-control) or shRNA directed against PI3K-C2β (Sh-PI3KC2β) were submitted to immunoblotting with anti-PI3KC2β antibody as indicated. Quantifications by densitometric analysis of the western blots are shown and are mean±SEM of 6 independent experiments. (2) Graph quantification of PI3P mass assay with hCMEC control (sh-Control) or PI3K-C2β knocked-down (sh-PI3K-C2β) cells showed that PI3KC2β was responsible of PI3P production. (B) PI3K-C2β knocked-down reduces inflammation-associated endothelial permeability—Confluent endothelial hCMEC cells transduced with shRNA control (Sh-control) or shRNA directed against PI3K-C2β (Sh-PI3KC2β) were cultured on transwell and stimulated with TNFα (25 ng/ml) over the time. The transendothelial electrical resistance (TEER) was measured with a voltohmeter Millicell ERS-2. Data are shown as mean±SEM (n=3). ***P<0.001; **P<0.01; *P<0.05, significance differences from control. (C) PI3K-C2β knocked-down maintains VE-cadherin to endothelial cell (EC) junctions in response to TNFα—Graph quantification of VE-cadherin immunoreactivities undertaken in hCMEC control (sh-Control) or knocked-down for PI3K-C2β (sh-PI3K-C2β) under basal conditions and after activation by TNFα (25 ng/ml) during 24 hours. Quantification further confirmed disrupted EC junctions in Sh-Control hCMEC cells 24 h after TNFα stimulation whereas VE-cadherin accumulated in PI3K-C2β knocked-down EC (sh-PI3K-C2β). Data represent mean±SEM (n=5), ***P<0.001; **P<0.01; *P<0.05.

### EXAMPLE

### Methods

### Mice

PI3K-C2β^{D1212A/D1212A} knock-in mice and wild-type littermates bred on a C57BL/6 background were generously provided by B. Vanhacsebroeck (Alliouachene, S et al. Cell Reports 13 (9), 2015). All experiments were performed on 8- to 12-weeks-old mice, unless otherwise specified, and housed in Anexplo vivarium (US006/Regional center of functional exploration and experimental resources, Inserm/Université Paul Sabatier, Toulouse, France). Animals' procedure were approved by the institutional animal care and use committee (CEEA-122 2014-54) and conduced in accordance with the guidelines of the national institute of health.

### Generation of Bone Marrow Chimeric Mice

The recipient mice were irradiated to the non-invasive exploration platform located at the Nuclear Medicine Department of the Rangueil Hospital (Biobeam Biological Irradiator 8000). The animals received a single dose of 9 Gray (Gy) for 6 min and their immune system rescued by bone marrow transplantation from either WT or PI3K-C2β KI donors after 24 h in ventilated cages with drinking water supplemented with 10% antibiotics Baytril (Bayer). The tMCAO surgery was performed approximately 4 weeks later.

tMCAO Versus Thromboembolic Stroke Mice Model

To investigate the functional role of class II PI3K-C2β in reperfusion injury induced by ischemic stroke we use the mechanical mouse model of tMCAO and the model of thromboembolic stroke. These two models provide powerful experimental approaches for translational stroke research and are representative of two different clinical situations. The first model results in prompt recirculation, mimicking cerebrovascular surgery or interventional thrombectomy, whereas the second mimics the cellular and molecular mechanisms of thrombosis and thrombolysis with tissue-type plasminogen activator (rt-PA), resulting in the gradual restoration of the recirculation. These two models provide powerful experimental approaches for translational stroke research and are representative of the two different clinical situations.

Transient Middle Cerebral Artery Occlusion (tMCAO)

Mice were anesthetized with 3% isoflurane in a mixture of 70% N2O/30% O2 for cerebral focal ischemia-reperfusion induction by tMCAO according to the established procedure Bracuninger et al., Methods Mol Biol. 2012; 788:29-42). After midline neck incision, the internal carotid artery was occluded with an 18-mm length of 4-0 nylon monofilament with a flame-rounded tip to occlude the origin of the Middle Cerebral Artery (MCA). After 1 h occlusion, mice were reanesthetized, the suture and ligatures were removed to initiate reperfusion for 24 h. Successful induction of focal ischemia was confirmed by contralateral hemiparesis. Exclusion criteria were excessive bleeding or death within 24 h after tMCAO.

### Thromboembolic Stroke

Mice were anesthetized with isoflurane (4-5% for induction, 1-2% thereafter) in a 70% N2O/30% O2 gas mixture. Thereafter, they are placed in a stereotaxic frame, the skin between the right eye and the right ear is incised, and the temporal muscle is retracted. A small craniotomy is performed, the dura is excised, and the middle cerebral artery (MCA) exposed. The pipette (glass micro-pipette, tip size 30-50 μm) is introduced into the lumen of the artery and 1 μL of murine α-thrombin (Haematologic Technologies Inc., Stago BNL, NL) is injected to induce in situ clot formation (Orset C, Stroke. 2007; 38(10):2771-2778). The pipette is not removed for 10 min after the injection of thrombin to allow the clot stabilization. The rectal temperature is maintained at 37±0.5° C. throughout the surgical procedure using a feedback-regulated heating system. Cerebral blood flow velocity (CBFv) is used as an occlusion index (blood flow is reduced by up to 60% of baseline) and is monitored using a laser Doppler within the MCA territory on the dorsal face of the skull over 60 min. These experiments were performed in the Experimental Stroke Research Platform (ESRP, Caen, France).

### In Vivo Brain Imaging

In vivo brain imaging is performed in the Biomedical Imaging Platform (Cyceron, Caen, France) using a 7t MRI (Brucker, pharmascan) on anesthetized mice (2% isoflurane in a 70% nitrous oxide and 30% oxygen mixture), 24 h post-occlusion. For this purpose, a set of sequences in the axial plan including time-of-flight angiography, T2-weighted (T2W), and T2*-weighted (T2*W) imaging will be performed. These sequences allow the assessment of arterial recanalization, ischemic infarction, and brain hemorrhages, respectively. Images are then post-processed using imageJ software for ischemic calculation and angiographic score measurements.

### Evans Blue Extravasation

The integrity of the blood brain barrier (BBB) was assessed by measuring extravasation of Evans blue dye into the brain parenchyma. A 2% solution of Evans blue in saline was injected intravenously at 4 mL/kg 1h after induction of tMCAO. Twenty four hours later, mice were anesthetized with isoflurane and perfused with saline through the left cardiac ventricle until infusion fluid was colorless. Mice were sacrificed, brains were removed and 2-mm coronal sections were sliced for photography.

### Immunohistochemistry

Front and rear portions of each brain that were postfixed for 48 hours at 4° C. in 10% neutral buffered formalin (Sigma), embedded in paraffin, and sectioned at a thickness of 10 μm. Tissue sections were mounted on pretreated slides and deparaffinized in xylene. Hematoxylin-and-eosin (HE) staining was performed on selected sections from each brain to assess the degree of leukocyte infiltration.

### RNA Extraction and Reverse Transcription

Tissues were homogenized and total RNA were extracted in Trizol reagent (Life Technologies, Gaithersburg, MD, U.S.A.) according to the manufacturer's suggested protocol. Total RNA concentration was determined from spectrophotometric optical density measurement (260 and 280 nm). Reverse transcriptase reactions were then carried out using the RNA PCR Core Kit (GeneAmp RNA PCR Core kit, ThermoFischer Scientific). Experiments were realized according to the manufacturer's suggested protocol and were carried out in a DNA Thermal Cycler 480 (Perkin Elmer, Branchburg, NJ, U.S.A.). The cDNA was then stored at −20° C.

The cDNA sequences for RPS29 (ribosomal protein small subunit 29), interleukin-1β (IL-1β), IL-6 and tumor necrosis factor-α (TNF-α) were obtained from GeneBank. The primer and probe sequences used are reported in Table 1. Real-time PCR was performed using the TaqMan Universal PCR Master Mix. All samples were run in duplicate and the output level reported as the average of the two duplicate. Amplification conditions were performed using ABI PRISM 7700 sequence detection system (PE Applied Biosystems). The threshold cycle, which represents the PCR cycle at which an increase in reporter fluorescence above background is first detected, was determined by the software, based on the standard curves.

Using the formula provided by the manufacturer (PE Applied Biosystems) and described by Wang et al. (Wang et al. Journal of Neuroscience Research, 2000; 59:238-246, Wang et al. J Cereb Blood Flow Metab, 2000; 20:15-20), the values were extrapolated to calculate the relative number of mRNA copies as compared with RPS29 levels as control. The data are presented as the mean±SD. ANOVA followed by Tukey post hoc analysis was used to evaluate differences between time points. Student's 1-tests were used to evaluate differences between left and right hemispheres.

### Cell Culture

Immortalized human brain capillary endothelial cells (hCMEC/D3 cell line), which retain the characteristics of the cerebral circulation (Weksler, B. B. et al. The FASEB Journal, 2005, 19, n° 13:1872-74.), were cultured in rat tail collagen I (Cultrex, Trevigen, France) coated plates (1.5 mg/mL) in medium consisting of EndoGRO medium (Merck Millipore) supplemented with a dedicated supplement (EndoGRO MV Supplement Kit, Merck Millipore), 1 ng/mL basal Fibroblast Growth Factor (Sigma-Aldrich) and 1% penicillin-Streptomycin (Invitrogen). Cells were cultured in an incubator at 37° C. with 5% CO₂ and saturated humidity. From these cells, a batch having integrated a vector by lentiviral transduction was created. HCMEC/D3 pLKO-ShRNA PI3KC2β (shRNA-PI3K-C2β) cells having integrated a shRNA directed against PI3K-C2β. For the cells transduced by the shRNA-PI3K-C2β or shRNA-control lentiviruses the medium was supplemented with 3 μg/mL puromycin. The cells were passed twice a week with Trypsin/EDTA (Sigma-Aldrich) or accutase (BD Pharmingen). The cells were counted using a cell counter (Z1 coulter particle counter Beckman Coulter Brea USA).

### Trans-Endothelial Electrical Resistance (TEER)

For trans-endothelial electrical resistance hCMEC/D3 were seeded on type I collagen pre-coated Transwell-Clear filters (Costar, Corning Incorporation). Assay medium was changed after 4 and 7 days and transport assays were performed when cells form monolayers (7-10 days after seeding). Culture systems on inserts were exposed to treatment (hrTNFα at 25 ng/ml), and TEER were measured using an epithelial volt-ohmmeter (Millicell). The resistance of ECM-coated inserts was used as control. The values obtained were plotted on GraphPad software and checked for significance.

### Western Blot Analysis

Proteins were extracted from tissues in lysis buffer containing 150 mM NaCl, 20 mM Tris·HCl pH7.4, 1% Triton X-100, 0.2% SDS, 4 mM EDTA, 10 μg/ml leupeptin, 10 μg/ml aprotinin, 1 mM Na3VO4, 1 mM PMSF. The homogenate was cleared by centrifugation at 4□C for 20 min at 13,000 g and the supernatant fraction recovered. Protein concentration was determined by colorimetric assay (BCA, Pierce). Homogenates were resolved by SDS-PAGE, transferred to nitrocellulose membranes and probed with antibodies to PI3K-C2β (1:1000) from BD Biosciences (#611342) overnight at 4° C. Antigen-specific binding of antibodies was visualized by ECL.

### Immunofluorescence

Cells were seeded at 2.5×10⁴ cells·cm⁻² in collagen I-coated glass coverslip in 24 well plates. After snap wash in PBS, cells were fixed in 4% formaldehyde and permeabilized with 0.1% Triton X-100. Cells were blocked in PBS with 1% BSA fatty acid-free 1 h and incubated with VE-Cadherin primary antibody (#555661, BD Pharmingen) in blocking solution 2 h at RT in humid chamber. After washes, lamellae are incubated with the appropriate fluorescent secondary antibody and DAPI to evaluate cell number. Coverslips were mounted on glass slides with Mowiol mounting solution. Confocal images were captured with a LSM780 operated with Zen software (Carl Zeiss). Profiling of fluorescence intensity was carried out with ImageJ (National Institute of Health, Bethesda, MA, USA).

### Mass Assay

PI3P levels were quantified by a mass assay as previously described (Chicanne, G. et al. Biochemical Journal. 2012, 447, n° 1:17-23). Preparation of cell extract for mass assay was as follows. After removing media, cells were immediately scraped off and recovered in ice-cold 1M HCl, followed by centrifugation at 2000 rpm at 4° C. and snap-freezing of the cell pellet. Samples were stored at −80° C. before processing for PI3P mass assay.

### Statistical Analysis

All data are shown as mean+/−S.E.M. The statistical significance of differences between means was calculated by one-way anova, two-way anova or t-test analysis, as appropriate. Statistical significance was assumed at p<0.05 and indicated as *p<0.05, **p≤0.01, ***p≤0.001 realize using Prism Software (GraphPad, version 5).

### Results

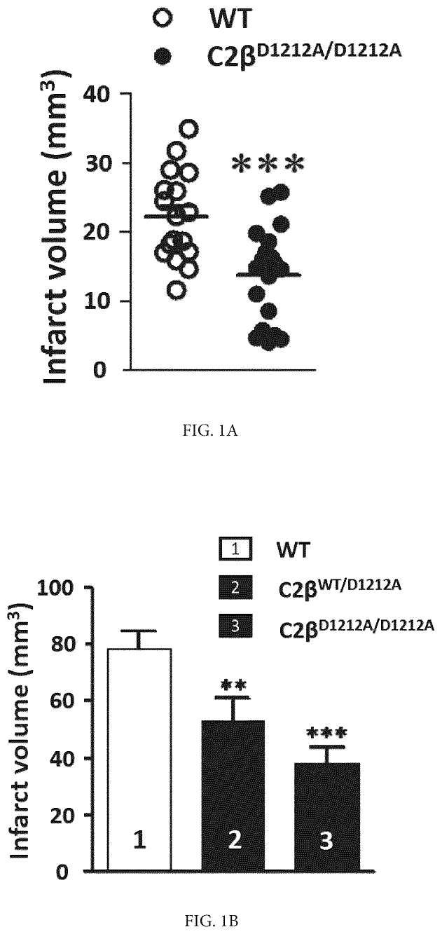

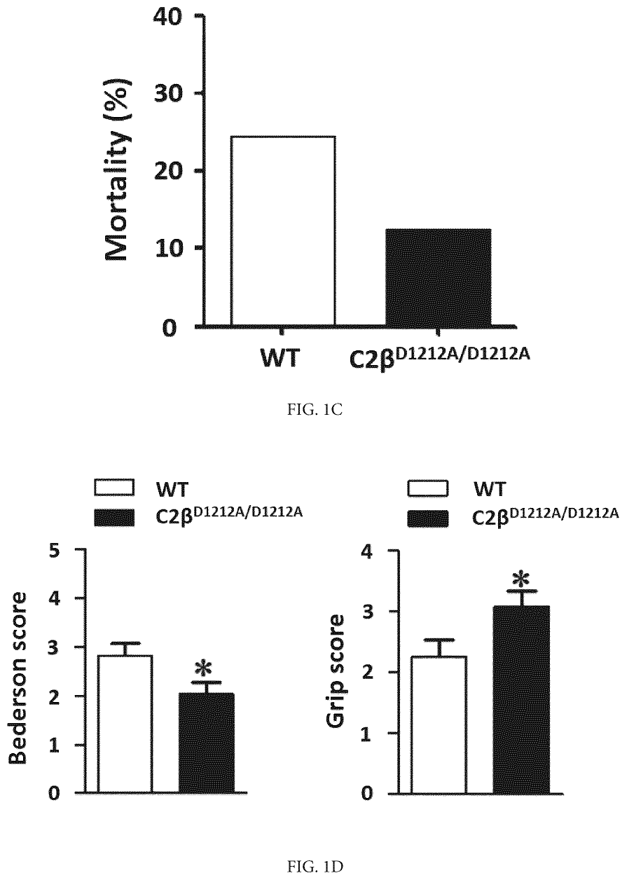

The results are depicted in FIGS. 1-4.

Firstly, the results show that genetic inhibition of PI3K-C2β reduces the cerebral infarction in two ischemia/reperfusion (I/R) models and improves neurological outcome. C2β^{D1212A/D1212A} mice displayed a significantly improved outcome compared to WT mice resulting in a significant increase in survival, a better overall neurologic function 24 hours after tMCAO (Bederson score: mean, 2.82 for WT vs 2.04 for C2β^{D1212A/D1212A}; P<0.05) and an improved motor function and coordination (grip test score: mean, 2.26 for wild-type vs 3.07 for C2β^{D1212A/D1212A}; P<0.05) (FIG. 1). Collectively, these data demonstrate that the marked reduction of infarct volume in C2β^{D1212A/D1212A} mice was functionally relevant.

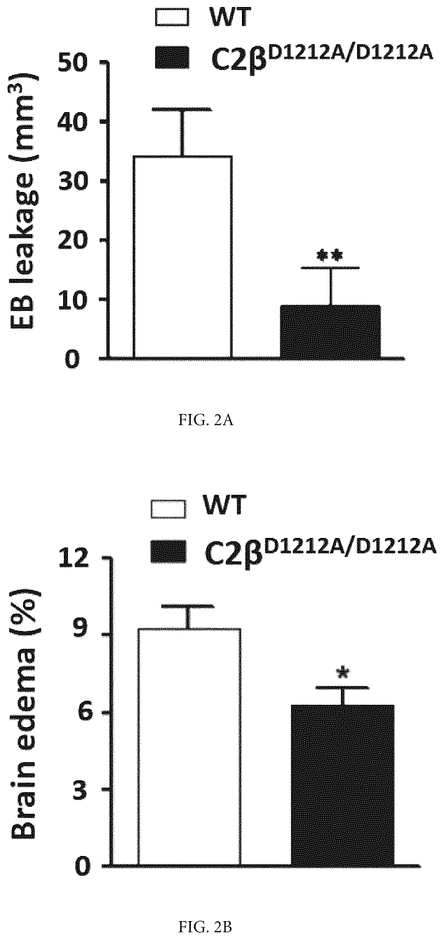

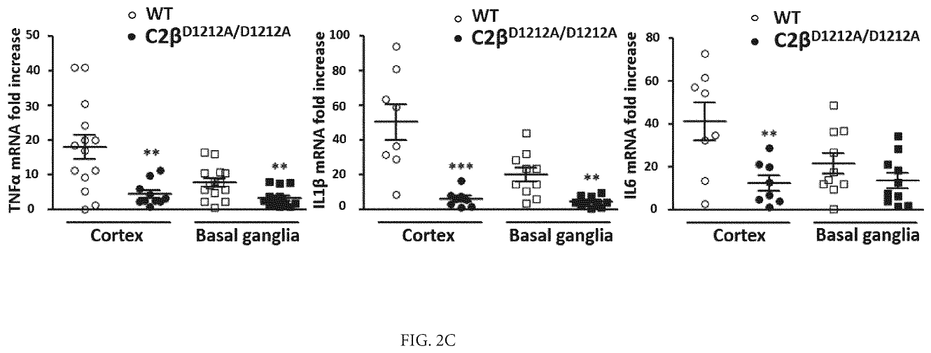

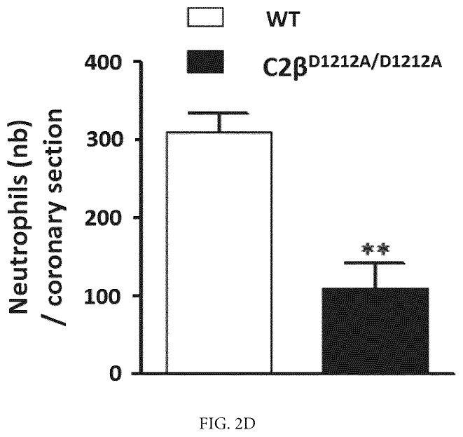

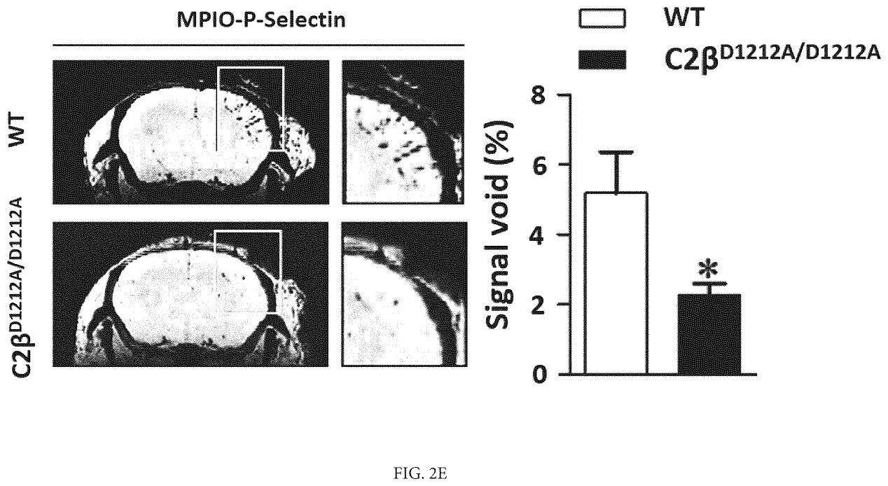

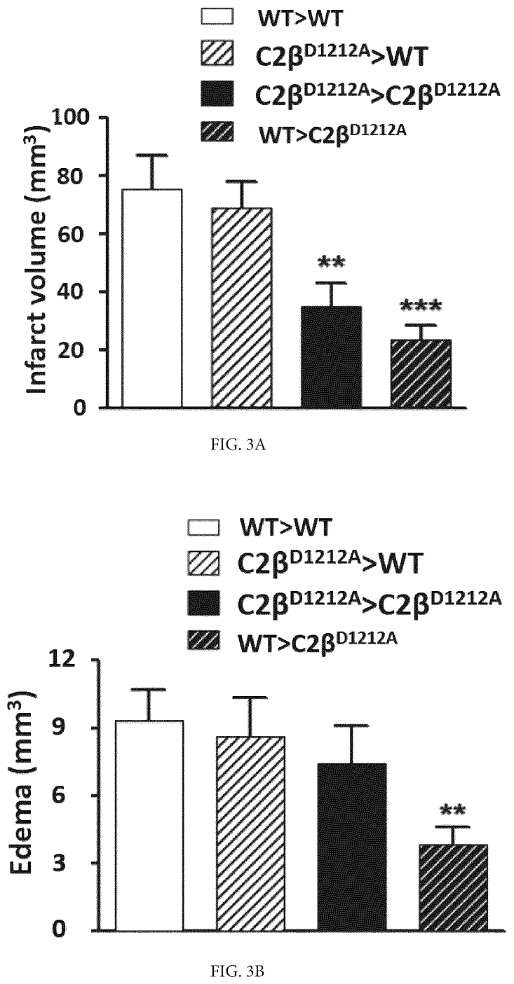

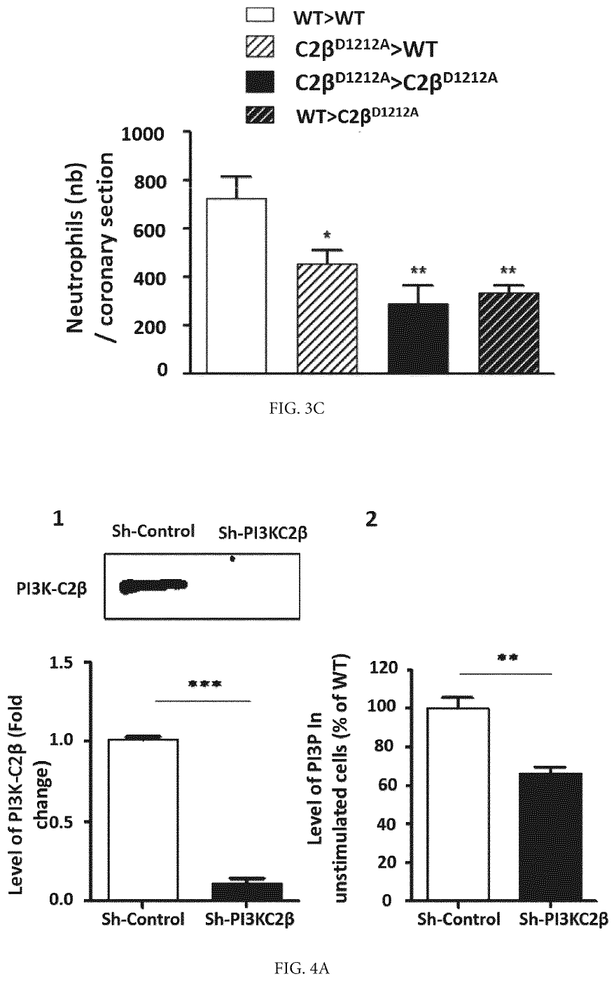

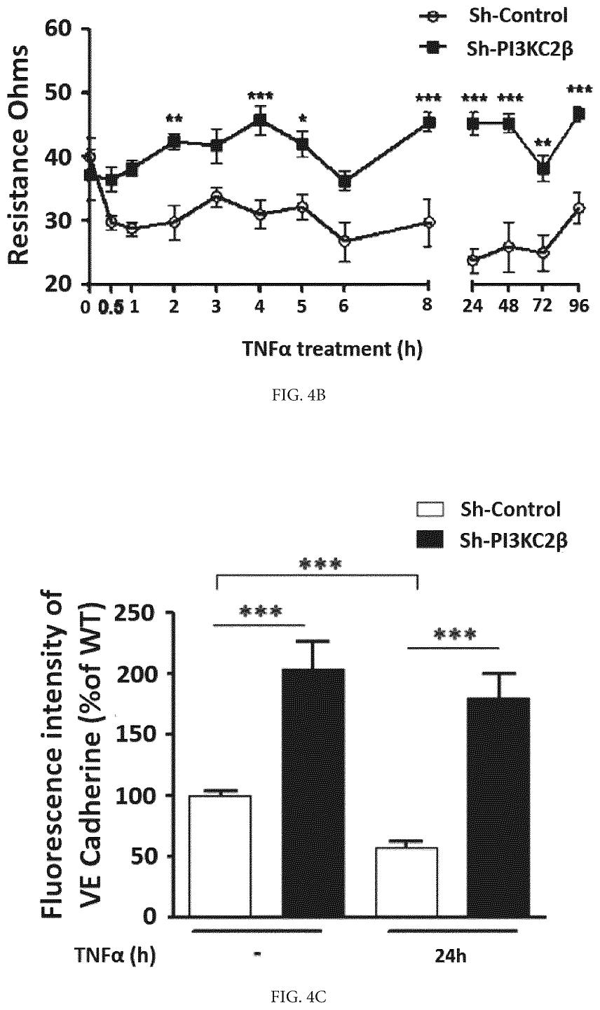

Genetic inhibition of PI3K-C2β stabilizes the blood-brain barrier (BBB after ischemic stroke and reduces inflammation. In the thromboembolic stroke model, ultrasensitive molecular MRI of cerebrovascular inflammatory molecules expressed by endothelial cells, such as adhesion molecule P-selectin, was used to evaluate the degree of brain inflammation in vivo. Antibody-based microsized particles of iron oxide (MPIOs) targeting P-Selectin were injected intravenously in mice 24 h after induction of acute thrombosis in the MCA. MRI was acquired 20 min after intravenous administration of targeted MPIOs. Absence of PI3KC2β activity (C2βD1212A/D1212A mice) efficiently protected from endothelial P-Selectin expression compared to WT mice (2.26% vs 5.18%) indicating a decrease of endovascular inflammation (FIG. 2). The results also show that inhibition of endothelial PI3KC2β reduces cerebral infarction, edema and neutrophils infiltration (FIG. 3). In human cerebral microvascular endothelial hCMEC/D3 cells, the results show that PI3K-C2β is critical for the regulation of PI3P level and PI3K-C2β knocked-down reduces inflammation associated endothelial permeability. PI3K-C2β knocked-down maintains VE-cadherin to endothelial cell (EC) junctions in response to TNFα (FIG. 4)

Altogether these results highlight the involvement of PI3K-C2β in infarct generation and CNS inflammation in two different models of stroke and demonstrate that inhibition of this lipid kinase is beneficial in acute ischemic stroke.

### REFERENCES

Throughout this application, various references describe the state of the art to which this invention pertains. The disclosures of these references are hereby incorporated by reference into the present disclosure.

1. A method for screening substances for use in preserving vascular endothelial cell barrier integrity, comprising:
providing a PI3KC2β protein;
contacting the PI3KC2β protein at least one test substance; and
selecting as a candidate substance an at least one test substance that decreases kinase activity of PI3KC2β in comparison to a negative control that is not contacted with the test substance.

2. The method of claim 1 wherein the PI3KC2β protein has an amino acid sequence according to SEQ ID NO. 1

3. The method of claim 1 wherein the PI3KC2β protein has an amino acid sequence identical to at least a partial length of SEQ ID NO. 1, wherein the partial length has kinase activity.

4. The method of claim 1 wherein the at least one test substance is a small organic molecule.

5. The method of claim 1 wherein the at least one test substance is selected from the group consisting of peptides, peptoides, circular or liner oligomeric compounds, benzodiazepine, hydantoin, biaryls, carbocyclic and polycyclic compounds, carbohydrate and amino acid derivatives, dihydropyridine, benzhydryl and heterocyclic compounds.

6. A method for screening substances for use in treating a condition in a patient in need of preserving vascular endothelial cell barrier integrity in said patient, comprising:
providing a PI3KC2β protein;
contacting the PI3KC2β protein at least one test substance;
selecting as a candidate substance an at least one test substance that decreases kinase activity of PI3KC2β in comparison to a negative control that is not contacted with the test substance; and
formulating the candidate substance in a pharmaceutical composition in an amount effective for treatment of the condition.

7. The method of claim 6 wherein the condition is ischemia caused by an ischemic-reperfusion injury.

8. The method of claim 6 wherein the pharmaceutical composition further comprises a thrombolytic agent.

9. The method of claim 8 wherein the thrombolytic agent is tissue-type plasminogen activator (t-PA).

10. The method of claim 6 wherein the PI3KC2β protein has an amino acid sequence according to SEQ ID NO. 1

11. The method of claim 6 wherein the PI3KC2β protein has an amino acid sequence identical to at least a partial length of SEQ ID NO. 1, wherein the partial length has kinase activity.

12. The method of claim 6 wherein the at least one test substance is a small organic molecule.

13. The method of claim 6 wherein the at least one test substance is selected from the group consisting of peptides, peptoides, circular or liner oligomeric compounds, benzodiazepine, hydantoin, biaryls, carbocyclic and polycyclic compounds, carbohydrate and amino acid derivatives, dihydropyridine, benzhydryl and heterocyclic compounds.


SEQUENCE LISTING (1 sequence)

SEQ ID NO: 1 - protein, 1634 aa, Homo sapiens
MSSTQGNGEHWKSLESVGISRKELAMAEALQMEYDALSRLRHDKEENRAKQNADPSLISWDEPGVDFYSKPAGRRTDLKLLRGLSGSDPTLNYNSLSPQEGPPNHSTSQGPQPGSDPWPKGSLSGDYLYIFDGSDGGVSSSPGPGDIEGSCKKLSPPPLPPRASIWDTPPLPPRKGSPSSSKISQPSDINTFSLVEQLPGKLLEHRILEEEEVLGGGGQGRLLGSVDYDGINDAITRLNLKSTYDAEMLRDATRGWKEGRGPLDFSKDTSGKPVARSKTMPPQVPPRTYASRYGNRKNATPGKNRRISAAPVGSRPHTVANGHELFEVSEERDEEVAAFCHMLDILRSGSDIQDYFLTGYVWSAVTPSPEHLGDEVNLKVTVLCDRLQEALTFTCNCSSTVDLLIYQTLCYTHDDLRNVDVGDFVLKPCGLEEFLQNKHALGSHEYIQYCRKFDIDIRLQLMEQKVVRSDLARTVNDDQSPSTLNYLVHLQERPVKQTISRQALSLLFDTYHNEVDAFLLADGDFPLKADRVVQSVKAICNALAAVETPEITSALNQLPPCPSRMQPKIQKDPSVLAVRENREKVVEALTAAILDLVELYCNTFNADFQTAVPGSRKHDLVQEACHFARSLAFTVYATHRIPIIWATSYEDFYLSCSLSHGGKELCSPLQTRRAHFSKYLFHLIVWDQQICFPVQVNRLPRETLLCATLYALPIPPPGSSSEANKQRRVPEALGWVTTPLFNFRQVLTCGRKLLGLWPATQENPSARWSAPNFHQPDSVILQIDFPTSAFDIKFTSPPGDKFSPRYEFGSLREEDQRKLKDIMQKESLYWLTDADKKRLWEKRYYCHSEVSSLPLVLASAPSWEWACLPDIYVLLKQWTHMNHQDALGLLHATFPDQEVRRMAVQWIGSLSDAELLDYLPQLVQALKYECYLDSPLVRFLLKRAVSDLRVTHYFFWLLKDGLKDSQFSIRYQYLLAALLCCCGKGLREEFNRQCWLVNALAKLAQQVREAAPSARQGILRTGLEEVKQFFALNGSCRLPLSPSLLVKGIVPRDCSYFNSNAVPLKLSFQNVDPLGENIRVIFKCGDDLRQDMLTLQMIRIMSKIWVQEGLDMRMVIFRCFSTGRGRGMVEMIPNAETLRKIQVEHGVTGSFKDRPLADWLQKHNPGEDEYEKAVENFIYSCAGCCVATYVLGICDRHNDNIMLKTTGHMFHIDFGRFLGHAQMFGNIKRDRAPFVFTSDMAYVINGGDKPSSRFHDFVDLCCQAYNLIRKHTHLFLNLLGLMLSCGIPELSDLEDLKYVYDALRPQDTEANATTYFTRLIESSLGSVATKLNFFIHNLAQMKFTGSDDRLTLSFASRTHTLKSSGRISDVFLCRHEKIFHPNKGYIYVVKVMRENTHEATYIQRTFEEFQELHNKLRLLFPSSHLPSFPSRFVIGRSRGEAVAERRREELNGYIWHLIHAPPEVAECDLVYTFFHPLPRDEKAMGTSPAPKSSDGTWARPVGKVGGEVKLSISYKNNKLFIMVMHIRGLQLLQDGNDPDPYVKIYLLPDPQKTTKRKTKVARKTCNPTYNEMLVYDGIPKGDLQQRELQLSVLSEQGFWENVLLGEVNIRLRELDLAQEKTGWFALGSRSHGTL